(19) United States
(12) Patent Application Publication
(10) Pub. No.: US 2015/0064675 A1
(43) Pub. Date: Mar. 5, 2015

(54) RESPONSIVE TOOL WITH SENSORS

(71) Applicants: Wade R. Eichhorn, Minneapolis, MN (US); Richard M. Duda, Stillwater, MN (US); Kristian G. Wyrobek, Minneapolis, MN (US)

(72) Inventors: Wade R. Eichhorn, Minneapolis, MN (US); Richard M. Duda, Stillwater, MN (US); Kristian G. Wyrobek, Minneapolis, MN (US)

(73) Assignee: 7-SIGMA, INC., Minneapolis, MN (US)

(21) Appl. No.: 14/015,951

(22) Filed: Aug. 30, 2013

(51) Int. Cl. G01L 1/20; G01L 1/18; A61B 19/00; G01L 1/04; G09B 23/28; G09B 5/00

(52) U.S. Cl. CPC G01L 1/20; G09B 23/28; G09B 5/00; A61B 19/46; G01L 1/04; G01L 1/18; A61B 2019/464
USPC 434/262; 73/862.629

(57) ABSTRACT

A flexible responsive surface of a tool is used on a tool (robotic or manual) that has a major surface and a sensor attached to and aligned with the major surface of the responsive tool. The sensor may have piezoelectric body, such as an elastic body containing conductive nanotubes homogeneously distributed therein to form a conductive path and at least two electrodes in electrical connection with the conductive path. Tools used in any surgical or medical treatment that are the subject of medical procedures are particularly useful in methods used in training personnel and evaluating medical techniques and medical personnel.

### BACKGROUND OF THE INVENTION

### 1. Field of Invention

The present invention relates to the field of sensors, particularly sensors that indicate local changes in conditions in or on articles, and more particularly in the field of positionable sensors that can be applied to a surface, embedded in or constructed within a device which expands or flexes under pressure. The invention also relates to flexible electrical sensors for use in various technologies including at least medical applications to provide information or measurement on the stress, elongation, pressure, or load that is applied to or placed upon the sensor. The present invention may be used as part of a device or system to provide information or measurement of stress, elongation, pressure, or load in the expansion of the device even in medical fields. In particular, the flexible nanotube composite sensor is bonded to or molded within an expandable and/or flexible elastomeric medical device system, such as a balloon (such as those delivered through catheters), to measure the performance of the device.

In more particularity, the sensors may be embedded in training or simulation devices that resemble, replicate or duplicate the appearance and properties of potions of anatomy, such as organs. These simulation devices may be used in training medical practitioners or forensic medical examiners in the simulated performance of specific procedures. The sensors can provide real-time feedback on the appropriateness of procedures and the propriety of forces, pressures and angles of contact during simulation of procedures.

### SUMMARY OF THE INVENTION

A flexible element (e.g., film, coating, patch, tube or strip) of elastomeric polymer containing from 0.02 to 8% by total weight of conductive nanoelements, particularly nanotubes, provides a particularly useful piezoresistive sensor. These sensors are attached to surfaces of or molded within the expandable or flexible elastomeric device, and measurements may be taken of changes in resistivity through or across the device (e.g., by measuring low voltage current across the strip) to determine changes in dimensions, stress and pressure on the strip. By having secure attachment to the surface of the expandable device distorts or flexes or pressures the sensors, or in such relationship to the surface that surface movement or having it molded within the expandable device, changes in the dimensions, pressure and stress on the device may be estimated with a significant degree of assurance of meaningful results.

The surface may in outside, inside (as in cavities or internal surfaces) or embedded between surfaces where sensing would be desirable. By using a simulacrum, replica, simulation, duplication, model (complete or partial) or replication of a region on which medical procedures (or other sensitive procedures, such as electronic repairs) would be performed, or models of devices on which procedures would be carried out, procedures may be simulated for purposes of training or improving skills or testing devices. These procedure simulations can be extremely sensitive and valuable in avoiding the need for training on live patient or subject or actually endangering expensive equipment.

### BRIEF DESCRIPTION OF THE FIGURES

FIG. 1 is a table showing a graphic representation of date relating physical properties of carbon nanotube silicone rubber composites within the generic scope of the present invention.

FIG. 2 is a graphic representation showing electrical resistivity properties of several carbon nanotube silicone rubber composites.

FIG. 3 is a graph showing the Dynamic Mechanical Analysis (DMA) Tan Delta (ratio between Storage and Loss Modulus) and nano-DMA testing of a carbon nanotube silicone rubber composite materials as presented in FIG. 1.

FIG. 4 graphically shows the piezoresitive response, measured by the change in current, of a flexible nanotube sensor as the flexible elastomeric device is inflated, in which the carbon nanotube sensor is molded.

FIG. 5 graphically shows the piezoresitive response, measured by the change in current, of the nanotube sensor as it expands along with the flexible elastomeric device and places pressure, up to 10 Newtons, on a very soft rubber material.

FIG. 6 shows an example of embodiment of a sensor within the generic scope of the present invention. FIG. 6 is a side sectional view of an electrically conductive polymer sensor 1 comprising of nanotubes to confer electrical properties.

FIG. 7 shows an example of embodiment of a sensor on a surface of an inflatable balloon element within the generic scope of the present invention.

FIG. 8 shows an example of embodiment of a sensor on a surface of an inflatable balloon element within the generic scope of the present invention.

FIG. 9 is a depiction of a Clasping Device, such as a tweezers, forceps, clamp or clasping device used in a robotic holding mechanism or manual device.

FIG. 10 is a second embodiment of a clasping device with a sensor component thereon, whereby a glove-like or surrounding structure is fitted onto or over the Clasping Device.

FIG. 11 is a depiction of a multilayer sensor whereby a metallic, or electrical conductive tool or probe, such as a scalpel or needle is inserted into a flexible substrate consisting of a highly electrical composite rubber or a dielectric material.

FIG. 12 is a representation of a medical training device wherein piezoresistive sensors are embedded within the body of the device.

FIG. 13 is the responsive force plot of the force of a staple applied to a synthetic vein of FIG. 12.

FIG. 14 is a sectioned view of a body which may represent a model of a body part such as an esophagus, colon or other organ into which a diagnostic or therapeutic probe may be inserted.

FIG. 15 is a graph of the dynamic response of a carbon nanotube sensor embedded within a rubber matrix as compared with the dynamic response of a dynamic mechanical analysis (DMA) measurement device.

### DETAILED DESCRIPTION OF THE INVENTION

The following definitions and descriptions are useful in understanding the scope of technology used in the practice of the present technology.

Nanocomposite Definition:

Nanomaterials that combine one or more separate components in order to obtain the best properties of each component (composite). In nanocomposite, nanoparticles (clay, metal, carbon nanotubes) act as fillers in a matrix, usually polymer matrix.

Nanomaterials Definition:

nanomaterials can be defined as materials which have structured components with at least one dimension less than 100 nm. Materials that have one dimension in the nanoscale are layers, such as a thin films or surface coatings. Some of the features on computer chips come in this category. Materials that are nanoscale in two dimensions include nanowires and nanotubes. Materials that are nanoscale in three dimensions are particles, for example precipitates, colloids and quantum dots (tiny particles of semiconductor materials). Nanocrystalline materials, made up of nanometre-sized grains, also fall into this category. Preferred dimensions for nanotubes are diameters of from 3 Angstroms, preferably at least 5 Angstroms, more preferably at least 10 Angstroms up to 100 nm, preferably up to 70 nm, more preferably up to 50 nm. Preferred ranges of diameters for nanotubes according to the present invention are from 0.5 nm to 30 nm.

Nanometer Definition:

One nanometer (nm) is equal to one-billionth of a meter, 10-⁹ m. Atoms are below a nanometer in size, whereas many molecules, including some proteins, range from a nanometer upwards.

Nanoparticle Definition:

Nanoparticles are particles of less than 100 nm in diameter. The preferred size range for diameters of nanotubes described above tends to be a preferred range for the largest dimension of nanoparticles also.

Nanotube Definition (Carbon Nanotubes):

Carbon nanotubes (CNTs) were discovered by Sumio Iijima in 1991. Carbon nanotubes are generally fullerene-related structures which consist of rolled graphene sheets, although multiple molecular level structures of nanotubes and variations in structure have been created and described. There are two generic types of CNT: single-walled (one tube) or multi-walled (more tubes). Both of these are typically a few nanometers in diameter and several micrometers to centimeters long.

Nanowires Definition:

Nanowires are ultrafine wires or linear arrays of dots, made from a wide range of materials, with nanodimension diameters. These are essentially extremely long nanotubes in some instances.

Simulation definition: As used in the practice of the present technology, a “simulation” is any article, device, organ, construction, model, body part (human or other animals), and the like which is used to provide an artificial device on which procedures may be practiced. These simulations may be considered as models, simulacrums, replications, copies, dummies, targets, and the like. The term “responsive model” shall generally be used as an inclusive term for the simulations having sensors associated therewith.

### Elastomeric Polymers

Elastomers are usually thermoset resins (requiring crosslinking or vulcanization) but may also be thermoplastic polymers. The polymer chains are cross-linked during curing, i.e., vulcanizing. The molecular structure of elastomers can be imagined as a ‘spaghetti and meatball’ structure, with the meatballs signifying cross-links. The elasticity is derived from the ability of the long chains to reconfigure themselves to distribute an applied stress. The covalent cross-linkages ensure that the elastomer will return to its original configuration when the stress is removed. As a result of this extreme flexibility, elastomers can reversibly extend (at least once, and preferably repeatedly without inelastic deformation occurring) from 5-700%, depending on the specific material. Without the cross-linkages or with short, uneasily reconfigured chains, the applied stress would more likely result in a permanent deformation. Temperature effects are also present in the demonstrated elasticity of a polymer. Elastomers that have cooled to a glassy or crystalline phase will have less mobile chains, and consequentially less elasticity, than those manipulated at temperatures higher than the glass transition temperature of the polymer. It is also possible for a polymer to exhibit elasticity that is not due to covalent cross-links. For example, crystalline polymers can be treated to alter their short range versus long range crystalline morphology to alter the elastic properties as well as other physical properties.

The present technology is related to U.S. patent application Ser. No. 13/599,935, filed Aug. 30, 2012, and to U.S. patent application Ser. No. 13/397,737 filed Feb. 16, 2012. Both references are incorporated by reference in their entireties herein.

Underlying technology within the scope of the present invention includes both sensors and methods of using sensors in processes or procedures. The novel articles used as sensors in the practice of the present technology comprise millimeter dimension (diameters and or three major dimensions between 0.2 to 100 mm) polymeric structures comprising from 0.2% to 8% by total weight of conductive nanotubes. The articles must have some degree of elastic deformation properties. For example, the article should be able to deform (bend, stretch, flex, extend, etc.) such that in at least one dimension (e.g., the length of a nanotube) there can be at least 5% total elastic deformation. That deformation could be measured from a base line 0 stress article with a return to that base line 0 stress (unstressed) length that has not inelastically changed by more than 0.5%. When used, the articles must have electrodes attached across the conductive dimension of the article, preferably aligned with the dimension of expected stress and elongation. Although the electrodes may be separated so as to extend perpendicularly or acutely or obtusely with respect to the expected dimension of elongation and stress, the peizoresistive effect is more accurately measured along a single dimension (or possibly along multiple directions, as the nanotubes often are not uniformly aligned, but may curl and twist into three dimensional form) parallel with the stress and elongation. The article may have electrodes fixed into the structure or may have attachment points for attaching the electrodes and placing them into contact with the conductive layer. The electrodes would extend to and be in electrical communication connection with a current or voltage measuring system. A voltage is applied across the conductive layer (the polymer-containing nanotubes) in the sensor, which may again be parallel with, perpendicular to or angled with respect to at least one dimension along which stress and elongation is expected during use, and the changes in the current (and/or voltage) is measured and the changes are correlated to stress and/or percentages of elongation in the article. As the current passed between sensors will change in a repeatable manner no matter what the orientation between the current flow and the elongation/pressure may be, a look-up table or other correspondence between the elongation/strain/pressure and changes in current can be established as a reference.

The flexible, elastic and/or expandable article, such as a strip or patch, may be secured to a surface or molded within an expandable elastomeric device that is to be manipulated or mechanically processed or chemically processed, where such processing or handling has surrounding concerns about changes in stress, dimensions, pressure or the like that can be measured by piezoresistive measurements. An elongate element, such as a sensor tube for example, may be a conductive nanotube-containing polymer of from 0.2 to 10 mm in diameter, and from 2 to 100 mm in length. A patch may comprise a square or rectangular OR oval or other geometric shape flat material comprising a conductive nanotube-containing polymer and two opposed edges. The electrodes are positioned at or about the opposed edges, the current is passed through the polymer, stress is applied to the patch, and the change in current is measured and correlated with amounts of stress and/or dimensional changes.

Various aspects of the invention include a piezoresistive sensor having an electrically conductive elastic body having at least one pair of opposed ends, and the elastic body containing conductive nanotubes homogeneously distributed therein, the elastic body having at least one surface with physical attaching elements thereon and the elastic body having electrodes attached at each of the at opposed ends. The conductive elastic body (that is the actual body of the sensor made from a composition) has an elastic range of between about 5% elongation and about 500% elongation. The conductive elastic body may have for example, from about 0.02% to 8% by total weight of the elastic body (not including electrodes) of conductive nanotubes. Preferably the conductive nanotubes are from about 0.2 to 5% by total weight of the conductive elastic body. The conductive nanotubes may be carbon nanotubes. The elastic body may be a polymer as described herein. The polymer may, by way of non-limiting examples, be selected from the group consisting of epoxy resins, silicone resins, ethylenically unsaturated elastomeric resins, and natural rubbers. The physical attaching elements are selected from the group consisting of polymers, chemical adhesives, adhesive tapes or mechanical attachments.

The present technology also includes a method of sensing dimensional changes, stress changes or pressure changes on a substrate including steps (not necessarily in the following order) of: non-destructively attaching a piezoresistant sensor to a surface of the device or molding the piezoresistant sensor within the device, the piezoresistant sensor comprising an electrically conductive elastic body having at least one pair of opposed ends, and the elastic body containing conductive nanotubes homogeneously distributed therein, the elastic body having at least one surface with two opposed ends and electrodes at each of the opposed ends, passing a current through the elastic body between the two electrodes, sensing the current passing through the elastic body, performing a mechanical step on the substrate, and measuring changes in the current between the electrodes. The measured changes are identified by an electronic look-up table or other execution of software by a processor receiving information/signals of the changes to identify changes in properties or conditions that are being monitored. The information may then be displayed on a video monitor if desired. The measured changes in current between the electrodes is related by execution of code in a processor to a pressure, stress level or change in dimension during performing of the expansion of the device mechanical step.

The invention also relates to a flexible and/or stretchable electrically sensor for use in any inflatable or flexible device on which stress or dimensional changes are to be determined, by way of non-limiting examples, tubes, balloons or coronary, vascular, orthopedic, and pelvic health applications and devices to provide information or measurement on the stress, elongation, pressure, or load that is applied to a expandable balloon medical device during, for example, a medical procedure or long term retention within the body.

The present invention may be described as a flexible substrate having a major surface and a sensor attached to and aligned with the major surface of the substrate, wherein:

the sensor comprises an elastic body containing conductive nanotubes homogeneously distributed therein to form a conductive path and two electrodes in electrical connection with the conductive path. At least two electrodes of the sensor may be in communication with both a power source and a processor. The sensor may be adhered to the major surface or embedded in the major surface. The major surface is preferably non-conductive. The major surface may comprise an elastomeric composition having a first modulus of elasticity and the elastic body of the sensor has a second modulus of elasticity and wherein the first modulus of elasticity is within 40% of the second modulus of elasticity. The major surface may be on an inflatable balloon having a conduit for transporting fluid into a cavity of the balloon to alter stress on the major surface of the inflatable balloon. The substrate may operate wherein presence of a nominally maximum fluid volume within the cavity maintains at least a 0.01 mm/m extension of a dimension in the elastic body of the sensor. The substrate may have the two electrodes of the sensor in communication with both a power source and a processor. The sensor may comprise an elastic body of a silicone rubber containing a loading of between 0.5% and 3%, by total weight of conductive nanotubes. The substrate may have the major surface as part of an inflatable balloon having a conduit for transporting fluid into a cavity of the balloon to alter stress on the major surface of the inflatable balloon. The substrate may be part of the major surface which is in turn an elastomeric composition having a first modulus of elasticity and the elastic body of the sensor has a second modulus of elasticity and wherein the first modulus of elasticity is within 40% or within 35% or preferably within 25% of the second modulus of elasticity. The major surface may be on an inflatable balloon having a conduit for transporting fluid into a cavity of the balloon to alter stress on the major surface of the inflatable balloon. The major surface may be on an expandable balloon element in a medical device that applies localized pressure in a patient. The sensor may comprise an electrically conductive silicone rubber composite comprised of a liquid silicone rubber with a multi-wall carbon nanotube loading of between 1%-3% by weight and a hardness between 10 and 60 Asker C hardness.

The invention may also include a method of detecting stress, pressure, contact or dimensional changes within an environment comprising positioning within the environment a substrate having a major surface and a sensor attached to and aligned with the major surface of the substrate, the sensor comprises an elastic body containing conductive nanotubes homogeneously distributed therein to form a conductive path and at least two electrodes in electrical connection with the conductive path;
applying a current across the sensor through one of the at least two electrodes;
determining changes in the current; and
providing signals indicating changes in the current to a processor; and
the processor executing code to correlate determined changes in the current to stress, pressure or dimensional changes in the sensor.
   These methods generally may use the substrates, sensors, devices and compositions described herein.

The devices and methods described and enabled herein may, by way of non-limiting examples include a responsive model having a major surface and a sensor attached to and aligned with (perpendicularly, parallel with, at predesigned angles, at the surface, under the surface or adjacent to) the major surface of the responsive model. The responsive model may have:

the sensor comprises an elastic body containing conductive nanotubes homogeneously distributed therein to form a conductive path and at least two electrodes in electrical connection with the conductive path. One of the at least two electrodes should be within the elastic body, and as shown later, both electrodes may be within the elastic body or one may be manipulated from an outside element or device and brought into contact with the at least one electrode within the elastic body. The at least two electrodes of the sensor are in communication (or in a communication link, when not actually communicating, as by wired connection or active/inactive wireless communication link) with both a power source and a processor. In the responsive model, the sensor may be adhered to the major surface or embedded in the major surface. Generally, in the flexible responsive model, the major surface is non-conductive, except for the sensor, or is at least not in a communication link with a power source or processor.

In one particularly advantageous configuration, the responsive model has the major surface attempt to replicate or simulate an interior or exterior surface on a responsive model in the shape of an organ of an animal. The organ may be any organ or structure within the body of the animal, including within the body of humans. It does not have to constitute the entire organ, as only those portions of the organ relevant to the medical procedure to be simulated need to be attached or present. For example, in creating a model of the heart, one does not have to include the entire vascular system. In a responsive model specifically designed for replacement of the myocardial valves, the pulmonary arties and veins need not be part of the model. Similarly, when creating a model for simulating operations on the duodenum, one may include (or not) the entire esophagus, but may usually exclude a replication of the mouth and jaws from the model. With other responsive models according to the present technology, discretion may be used in determining what degree or extent of replication needs to be done. It is, of course, possible to have a support system for carrying these partial replications of organs as responsive models. For example, in providing responsive models of the esophagus and trachea, a (responsive or non-responsive) model of a head and throat may be provided, the responsive model would then be inserted into the model of the head and throat (and even thorax), the practice medical procedure undertaken, the signals from contact, pressure, penetration, and other physical events determined and evaluated, and the training or testing procedure completed. If necessary or desirable, the used responsive model is then removed from the head and throat model, the responsive model cleaned, repaired or discarded, and the non-responsive model of the head and throat is then cleaned and may be reused.

In the responsive model, the major surface may be composed of the various materials described herein, including an elastomeric composition having a first modulus of elasticity and the elastic body of the sensor has a second modulus of elasticity and wherein the first modulus of elasticity is within 40% of the second modulus of elasticity. As suggested elsewhere herein, the major surface is on an interior surface of an opening or open pathway within a responsive model in the shape of an organ of an animal. Animals, of course, include human beings. The opening or open pathway may duplicate a shape and dimensions of natural openings and natural open pathways in a human body. The responsive model attempts to provide not only the major surface with physical properties that approximate physical properties of an organ or a structure within a human body, but also the general and overall physical properties of the entire responsive model attempt to duplicate or simulate approximate physical properties of an organ or a structure within a human body. Thus, cartilage, ligaments, bones, adjacent tissue and the like are also replicated in the model to create a maximum similarity to actual surgical conditions and materials. The responsive model may have the two electrodes of the sensor in communication with both a power source and a processor. The power source may be a single power source or there may be multiple power sources. In one preferred embodiment, the sensor may be constructed with an elastic body (e.g., by way of non-limiting examples of a silicone rubber, fluoro-elastomer, styrene-butadiene elastomer, natural rubber, latex rubber, etc.) containing a loading of between 0.5% and 3%, by total weight of conductive nanotubes. The range of compositions and contents may vary as described elsewhere within this disclosure. One specific embodiment of the responsive model includes a sensor of an electrically conductive silicone rubber composite which as the elastomeric or rubbery stretchable component, includes a silicone rubber with a multi-wall carbon nanotube loading of between 1%-3% by weight and a hardness between 10 and 60 Asker C hardness.

A method according to the present technology of detecting stress, pressure, contact, penetration or dimensional changes during a simulation of a medical procedure within an environment may include, for example, steps of positioning within the environment a responsive model having a major surface and a sensor attached to and aligned with the major surface of the responsive model, the sensor comprises an elastic body containing conductive nanotubes homogeneously distributed therein to form a conductive path and at least two electrodes in electrical connection with the conductive path;
applying a current across the sensor through one of the at least two electrodes;
simulating activity within the environment imitating activity occurring during the medical procedure;
determining changes in the current or voltage; and
providing signals indicating changes in the current to a processor; and
   the processor executing code to correlate determined changes in the current to stress, pressure, contact, penetration or dimensional changes in the responsive model comprising the sensor. The major surface my include an interior or exterior surface on a responsive model in the shape of an organ of an animal and one of the at least two electrodes is positioned within a moveable implement that is moved in at least two dimensions during the simulation of activity. The major surface may be on an interior surface of an opening or open pathway within a responsive model in the shape of an organ of an animal and the moveable implement containing the one of the at least two electrodes comprises a medical implement useful during a medical procedure.

In performing this aspect of the present technology, a medical tool, either functional or itself a partial or complete replica (e.g., by way of non-limiting examples, needles, scalpels, blades, catheters, syringes, stents, saws, lasers, implants [temporary or permanent], prostheses, struts, supports, tongs, medical pliers, pacemakers, defibrillators and the like) may carry at least one of the electrodes, such that a circuit to provide a signal is completed only by introduction of the medical tool into the responsive model environment. For example, the overall signals may be effectively neutral from the system, until the medical tool is brought into the procedural environment. Upon proximity to the sensors in the responsive models (e.g., a proximity sensor such as a light emitting and receiving transducer, magnetic field proximity sensor, etc.), contact with a surface that alters the position, tension, pressure, dimensions and/or distribution of or within the surface or body of the sensor (as generally described herein), moisture indicators/sensors (where the simulated organ is fluid-filled and sensing is of spurious penetration of the organ and release of liquid) and any other physical events that causes altered signals or responses from the sensors, the electrode in the medical tool act as one of the required at least two electrodes in the responsive model system. The practices of these methods, the opening or open pathway may duplicate shape and dimensions of natural openings in a human or other animal (for veterinary procedures) body and the medical implement is manipulated by direct manual control or robotic control of the medical implement in movements attempting to simulate the medical procedure on the responsive model.

In the use of robotic controls, these simulations are also very important. Pressures, forces and other physical alterations effected by the robotic extension(s) are not transmitted back to the operator. An operator has no sensory feedback on the degree of these physical events being administered during actual performance of procedures. An operator therefore may have no concept of whether applied forces are capable of crushing and otherwise damaging targeted materials or whether the forces applied are so minimal that even support of the target is not accomplished. By having measures sensor feedback, the forces can be monitored, measured and information related thereto is relayed to the operator (by measurements on a screen or printed sheet, by visual display on a video display screen, by visual replication or animation of the procedural events). For example, a animation of a cutaway view of the esophagus and trachea may be shown, and the effects of the robotically applied forces may be visually represented, along with or without additional warning information.

Such additional warning information might be alteration of the color of the visually provided image (which may be animation or direct imaging, with overlaid color control), audio content (e.g., warning sounds or buzzers at varying intensities indicating deviation from a desired range of forces. For example, excessive pressure may be indicated by one particular category of sounds, such as high-pitched buzzing, and insufficient pressures may be indicated by low-pitch humming), combinations of visual and audio effects, and even responsive sensory feedback. Haptic feedback may be provided from the determined sensor responses. In the use of robotics, for example, gloves or other fitted controls or manually directed control are used. By embedding haptic responsive technology into the controls, there can be direct feedback to the user. The haptic responses may be literal (that is the haptic responses attempt to literally replicate the forces being transmitted by the robotically controlled tool) or may be an enhanced response (where the response is amplified to better indicate minor variations in forces being robotically distally applied). For example, a high frequency tingling or vibration may be imparted at pressure levels outside desired tolerances, and low frequency vibration may be imparted by the haptic equipment at pressure or force levels below the desired range. Pressure changes in gloves (such as those pressure applied during blood pressure measurement cuffs) can also be used to provide a haptic response.

In performing the method, signals of determined changes correlated by the processor are provided by the processor in the form of image signals and the image signals are display in real-time on a visual display screen. The real-time response is of course important to the necessary feedback during training or testing.

The composition of the responsive model may be (but need not be, because they are not introduced into a body) biocompatible or non-biocompatible elastomeric material. Exemplary of the biocompatible polymer material used in forming the responsive models, the links or the stress concentrators includes the group of polymers consisting of polyurethanes, polyetherurethanes, polyesterurethanes, silicone, thermoplastic elastomer (C-flex), polyether-amide thermoplastic elastomer (Pebax), fluoroelastomers, fluorosilicone elastomer, styrene-butadiene rubber, butadiene-styrene rubber, polyisoprene, neoprene (polychloroprene), polyether-ether-ketone (PEEK), ethylene-propylene elastomer, chlorosulfonated polyethylene elastomer, butyl rubber, polysulfide elastomer, polyacrylate elastomer, nitrile rubber, a family of elastomers composed of styrene, ethylene, propylene, aliphatic polycarbonate polyurethane, polymers augmented with antioxidants, polymers augmented with image enhancing materials, polymers having a proton (HI) core, polymers augmented with protons (H+), butadiene and isoprene (Kraton) and polyester thermoplastic elastomer (Hytrel), polyethylene, PLA, PGA, and PLGA.

The responsive models may be part of systems and devices and the like used for treatments for many varieties of medical procedures in which the procedures may cause any contact, create pressure, increase volume restrictions, deliver materials, remove materials, stabilize organs, implant devices, surgically alter organs, and the like. Non-limiting examples of such procedures include at least treatment of vascular occlusions, gastric insertions, spinal stabilization, aneurism stabilization, drug delivery implants, joint stabilization, bone stabilization, organ stabilization, delivery of medical devices, infusion devices, penile implants, bladder control devices, intestinal controls, urethral implants, orthopedic implants, tracheotomy, by-pass surgery, transplants, tissue removal, tissue reconstruction and the like.

The following description of the Figures will further assist in an understanding of the present technology.

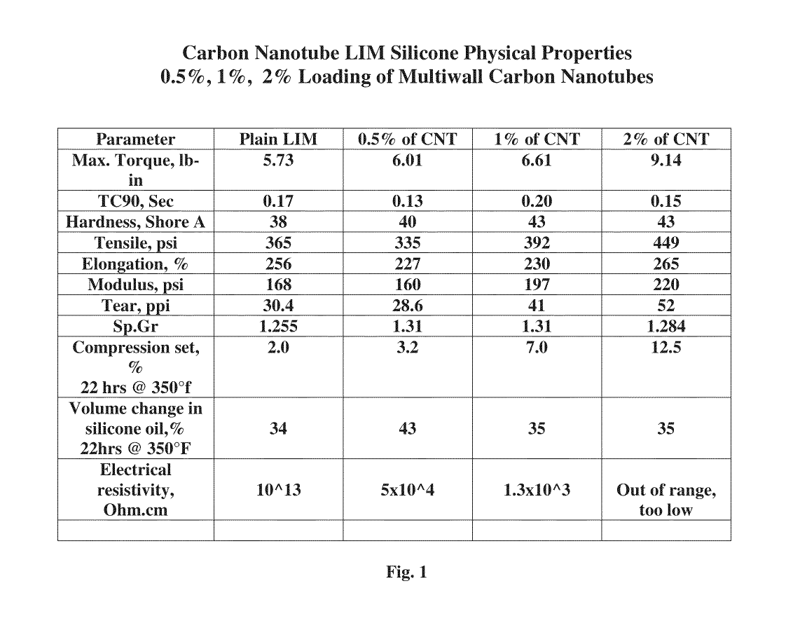

FIG. 1 is a table showing a graphic representation of date relating physical properties of carbon nanotube silicone rubber composites within the generic scope of the present invention. The table shows those properties of materials composed of a base-platinum-cured, liquid silicone composition curable to a rubber, the curable composition loaded with concentrations of 0.5%, 1% and 2% commercially available multi-wall carbon nanotubes.

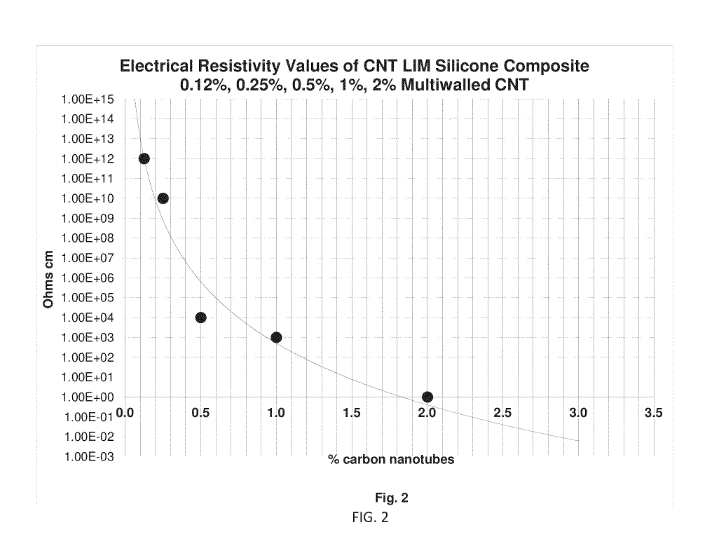

FIG. 2 is a graphic representation showing electrical resistivity properties of several carbon nanotube silicone rubber composites. Loading of 0.12%, 0.25%, 0.5%, 1.0% and 2.0% of commercially available multi-wall carbon nanotubes was added to a standardized composition of platinum cured liquid silicone rubber given in FIG. 1. Unless stated otherwise, the standard elastomer used in all examples (for convenience and to allow facile comparison of results only, a single composition was used, although not limiting the scope of the invention and presented with all data provided herein) was Shin Etsu X-34-1372, a two part, platinum cured liquid silicone rubber. The nanotubes were multiwall carbon nanotubes manufactured by Hyperion Catalysis and are approximately 4 nm in diameter by 1 micron or less in length.

The resultant electrical resistivity values, measured in Ohms cm, are plotted. The dramatic drop in electrical resistivity with very low loadings of carbon nanotubes is evident. The present invention may incorporate compositions displaying the electrical resistivity properties shown in FIG. 2 for a nanotube sensor, or other compositions, as generically described herein that display sufficient levels of resistance and piezoelectric resistivity as described herein.

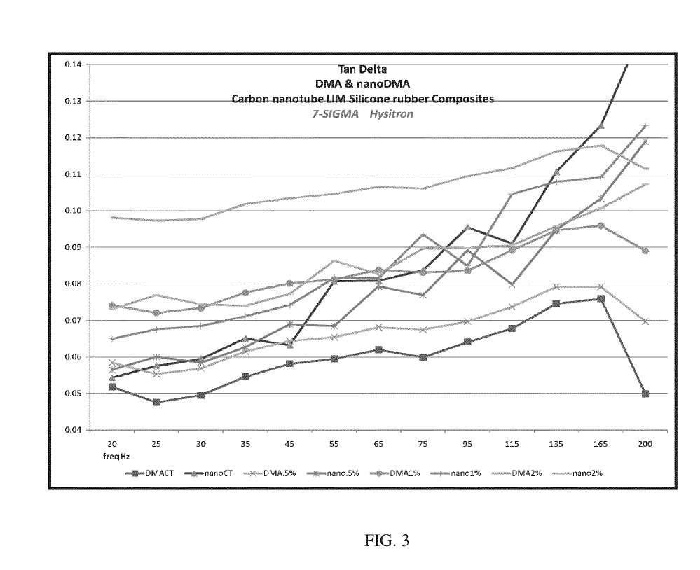

FIG. 3 is a graph showing the Dynamic Mechanical Analysis (DMA) Tan Delta (ratio between Storage and Loss Modulus) and nano-DMA testing of a carbon nanotube silicone rubber composite materials as presented in FIG. 1. The DMA plot is Tan Delta which is a ratio of the storage and loss modulus. Also are plotted a conventional DMA test with the nanoDMA testing. Dynamic Mechanical Analysis was carried out by Akron Research & Development Labs using a Visco Analyzer 2000 DMA150 in compression mode. Nanomechanical measurements were performed on a Hysitron TI 900 Tribolndenter™ tester by Hysitron, Inc. The graphically displayed results show the relationship between the DMA and the nano-DMA measurements of a frequency sweep from 20 to 200 hertz, and indicate a correlation of dynamic mechanical properties at the micro and nano levels of performance under strain. The indications are that the low loadings of carbon nanotubes within the general scope of the present invention (e.g., 0.5% to about 3% by total weight of the composition) does not adversely affect the mechanical performance of the material compared to the un-filled base material, thus preserving the physical properties of the chosen base polymer.

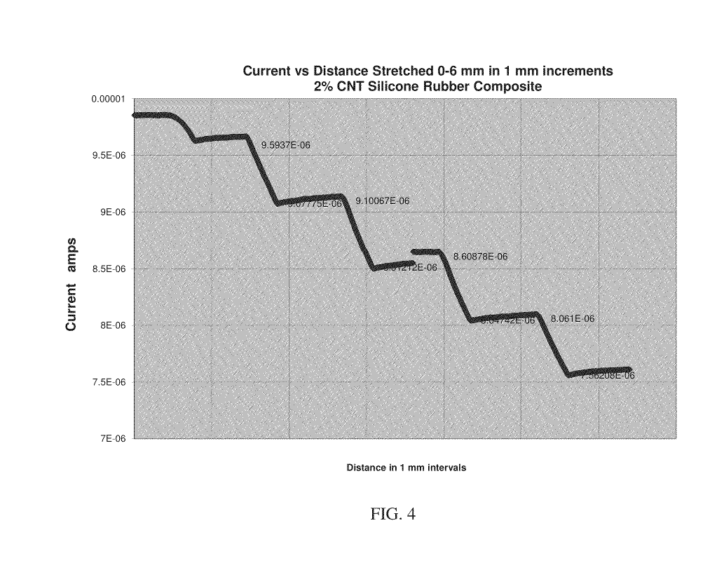

FIG. 4 shows the piezoresitive response, measured by the change in current, of a flexible nanotube sensor, composed of material chosen from, but not limited to, FIG. 2, as it is stretched as the flexible elastomeric device is inflated, in which the cnt sensor is molded.

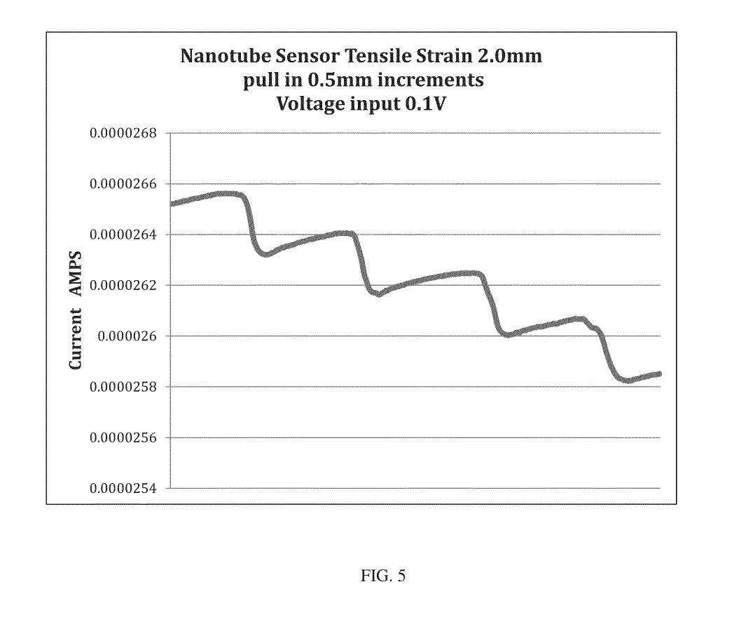

FIG. 5 shows the piezoresitive response, measured by the change in current, of the nanotube sensor, composed of material chosen from, but not limited to, FIG. 2 as it expands along with the flexible elastomeric device and places pressure, up to 10 Newtons, on a very soft rubber material.

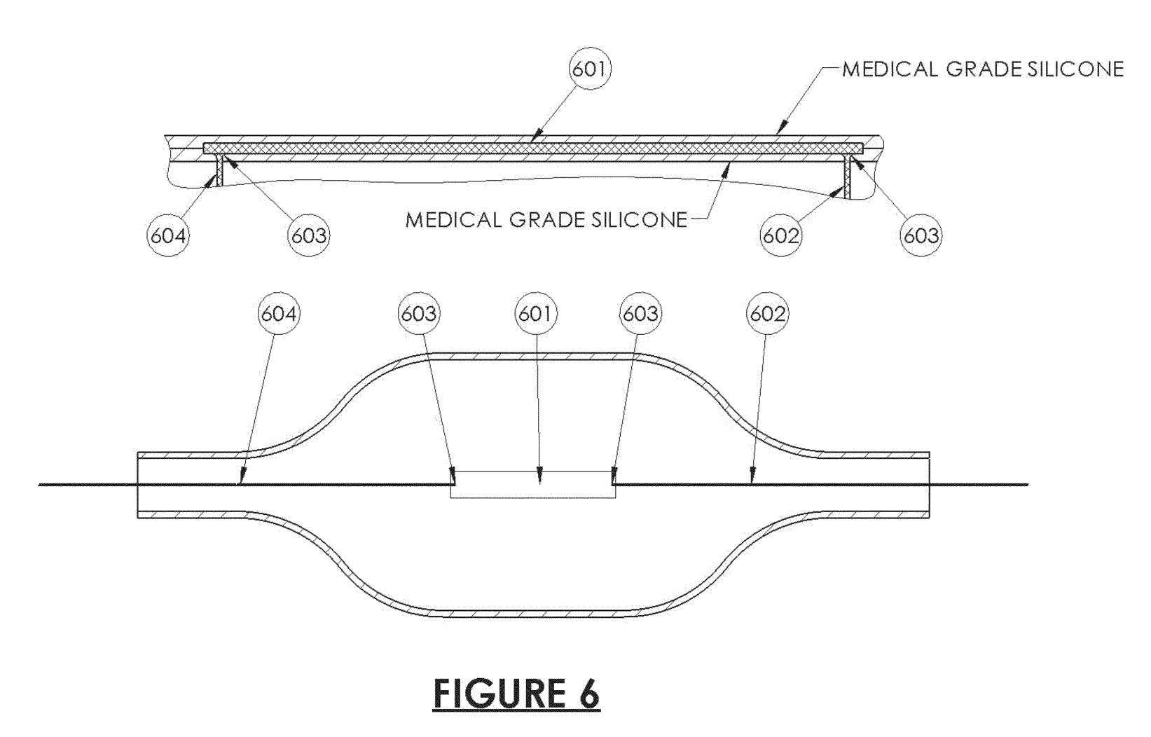

FIG. 6 shows an example of embodiment of a sensor within the generic scope of the present invention. FIG. 6 is a side sectional view of an electrically conductive polymer sensor 1 comprising of nanotubes to confer electrical properties. The sensor is comprised of the cured silicone polymer (or equivalent elastomer or flexible polymer). This is a flexible silicone rubber with carbon nanotube uniformly (essentially homogeneously, within the limits of real physical limits on the use of finite material) dispersed within the polymer at a preferred loading of between 0.5% and 3.0%. On each end of material 1 an electrical wire 2 and 4 (electrode) and connection 3 which are molded or affixed to the carbon nanotube rubber 1. The sensor is molded within the elastomeric medical balloon device, where a medical grade polymer encompasses the sensor. The sensor 601 is shown with its two leads 602 604 attached at points 603 embedded within medical grade silicone layers. A second embodiment is shown with the sensor 601, leads 602 604 and connection points 603 carried within the volume of an inflated balloon.

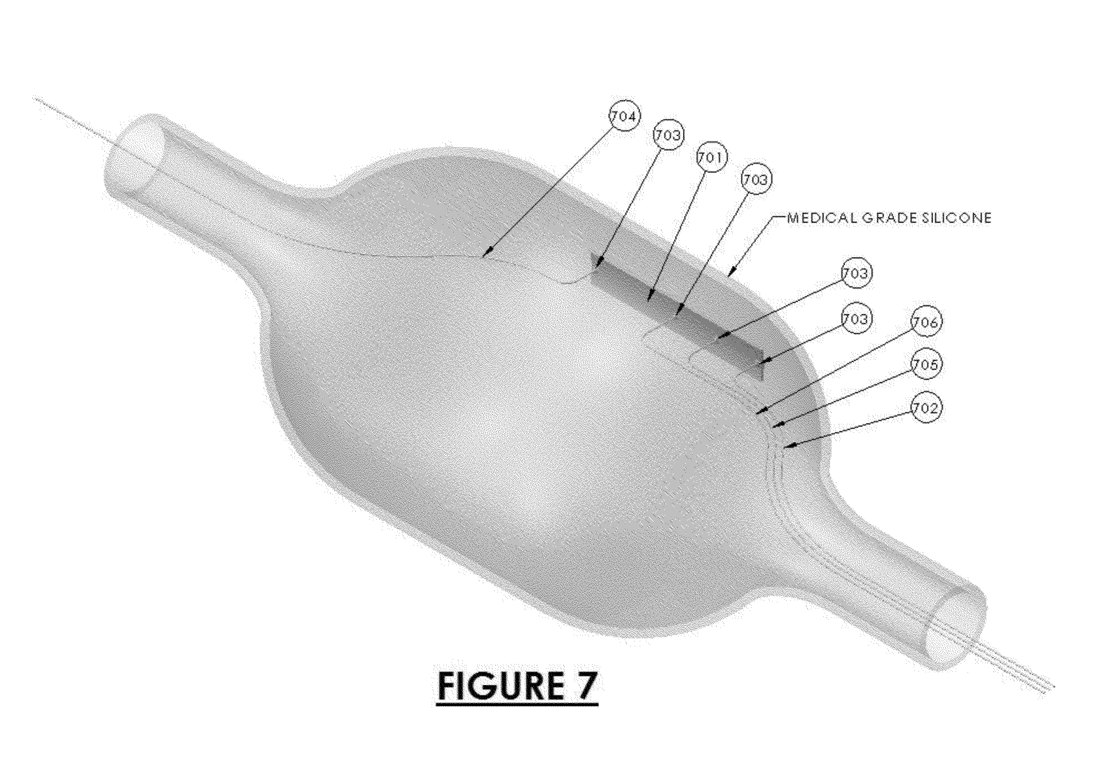

FIG. 7 shows an example of embodiment of a sensor within the generic scope of the present invention. FIG. 7 is a side sectional view of an electrically conductive polymer sensor 1 comprising of nanotubes to confer electrical properties. The sensor is comprised of the cured silicone polymer (or equivalent elastomer or flexible polymer). This is a flexible silicone rubber with carbon nanotube uniformly (essentially homogeneously, within the limits of real physical limits on the use of finite material) dispersed within the polymer at a preferred loading of between 0.5% and 3.0%. On each end of material 701 an electrical wire 702 and 704 (electrode) and connection 703 which are molded or affixed to the carbon nanotube rubber 701. Additionally, for example, between electrical wires 702 and 704 additional wires 705 and 706 may be applied, and connection 703 the sensor is molded within the elastomeric medical balloon device, where a medical grade polymer encompasses the sensor. The total number of wires connected to conductive polymer 701 may be 3 or more. The sensor is molded within the elastomeric medical balloon device, where a medical grade polymer encompasses the sensor.

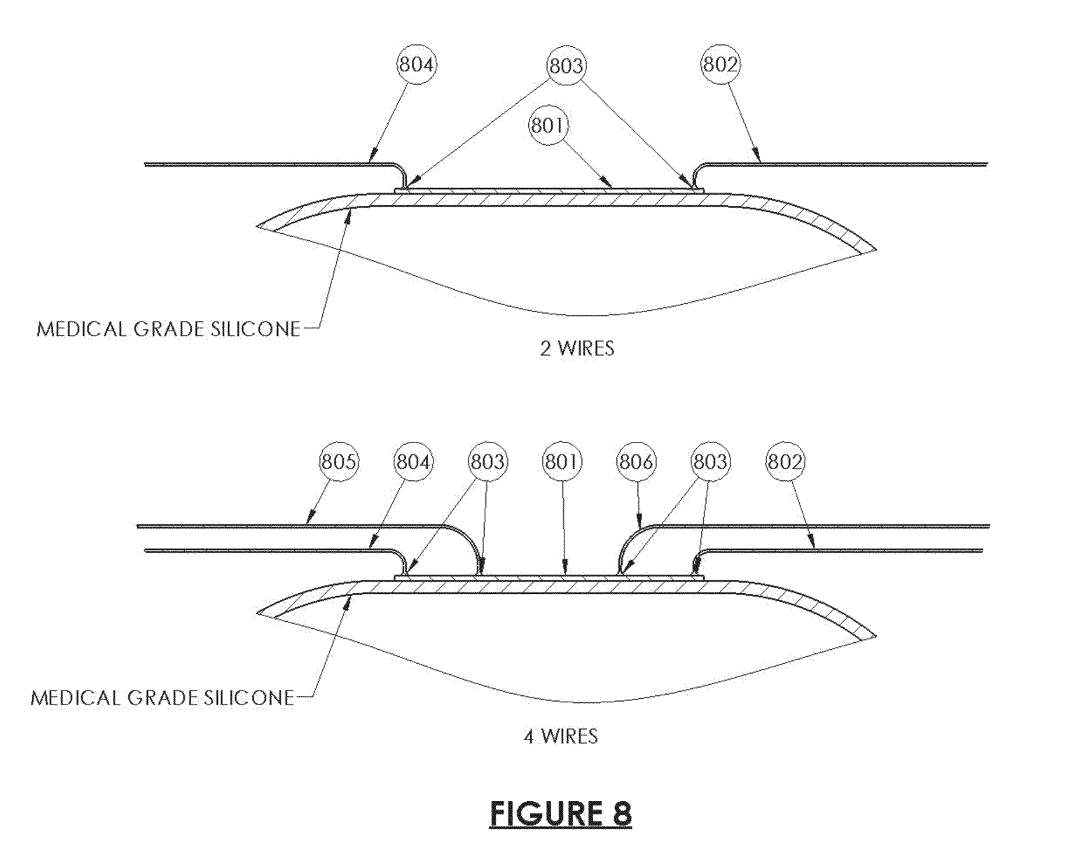

FIG. 8 shows examples of embodiment of a sensor within the generic scope of the present invention. FIG. 8 is a side sectional view of an electrically conductive polymer sensor 1 comprising of nanotubes to confer electrical properties. The sensor is comprised of the cured silicone polymer (or equivalent elastomer or flexible polymer). This is a flexible silicone rubber with carbon nanotube uniformly (essentially homogeneously, within the limits of real physical limits on the use of finite material) dispersed within the polymer at a preferred loading of between 0.5% and 3.0%. On each end of material 801 an electrical wire 2 and 4 (electrode) and connection 803 which are molded or affixed to the carbon nanotube rubber 801. Additionally, for example, between electrical wires 802 and 804 additional wires 805 and 806 may be applied, and connection 3 the sensor is molded within the elastomeric medical balloon device, where a medical grade polymer encompasses the sensor. The total number of wires connected to conductive polymer 801 may be 3 or more. The sensor is affixed to a surface, interior or exterior, of the elastomeric medical balloon device.

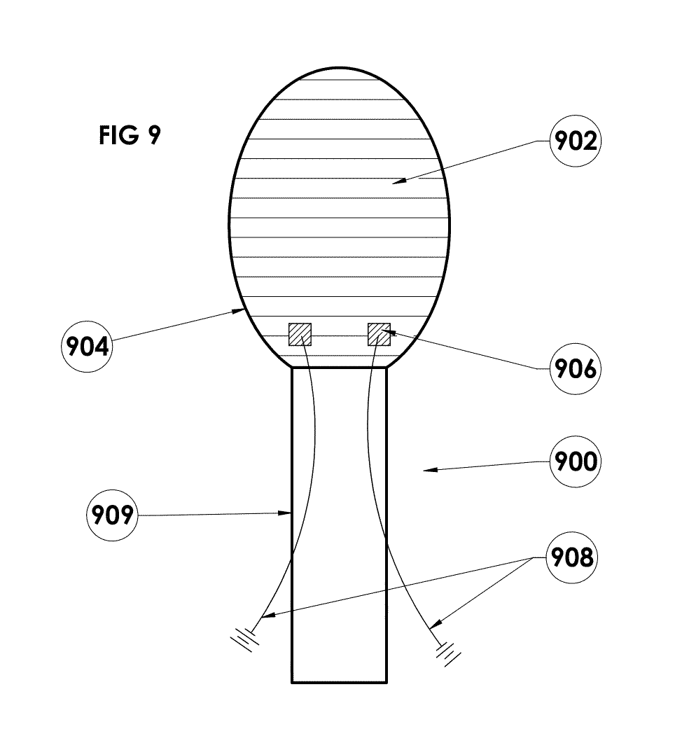

FIG. 9 is a depiction of a Clasping Device, such as a tweezers, forceps, clamp or clasping device used in robotic holding mechanism. Clasper 900 is comprised of a body 909 and an end clasping portion 904 to which is affixed a sensor body 902. To the sensor 902 are attached electrodes 906 that have attached electrical wires 908. The Clasping Deice may also be a manual tool, especially where used to train medical personnel in the appropriate levels of pressure during procedures. Although clasping devices are emphasized, any tool that applies pressure (e.g., pushes against surfaces to restrain or move tissue) can also be incorporated into this technology. Spreading tools, flattening tools (e.g., spatula-like tools) and the like may also be provided with sensor layers or components as described herein.

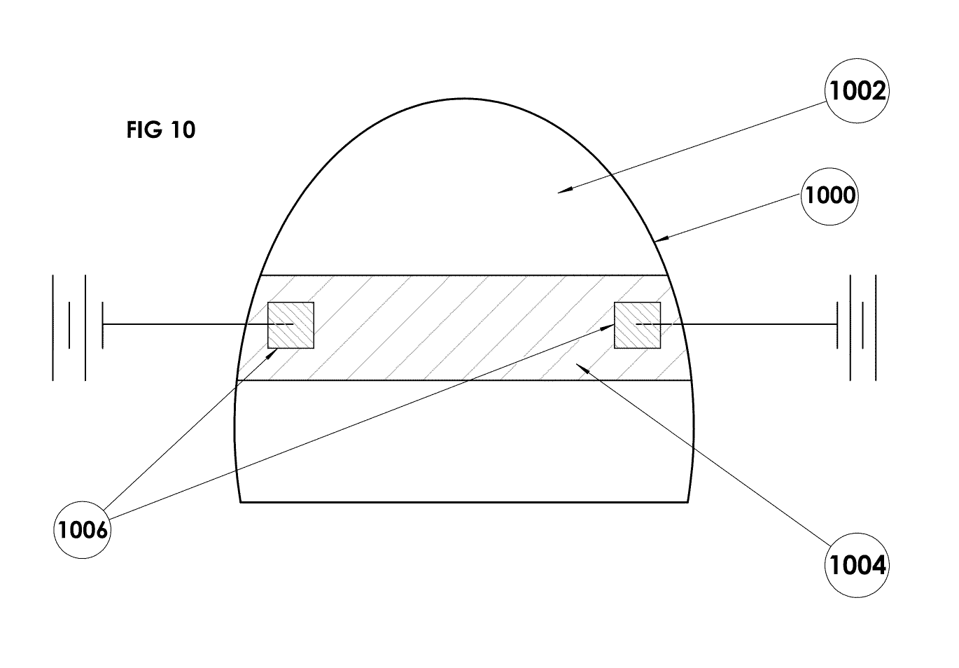

FIG. 10 is a second embodiment of a clasping device with a sensor component thereon, whereby a glove-like or surrounding structure 1000 is fitted onto or over the Clasping Device. The body of the surrounding structure 1000 may be comprised of a silicone rubber or other flexible electrically insulated polymer 1002, into which is embedded a sensor 1004. Two electrodes 1006 are affixed to sensor 1004.

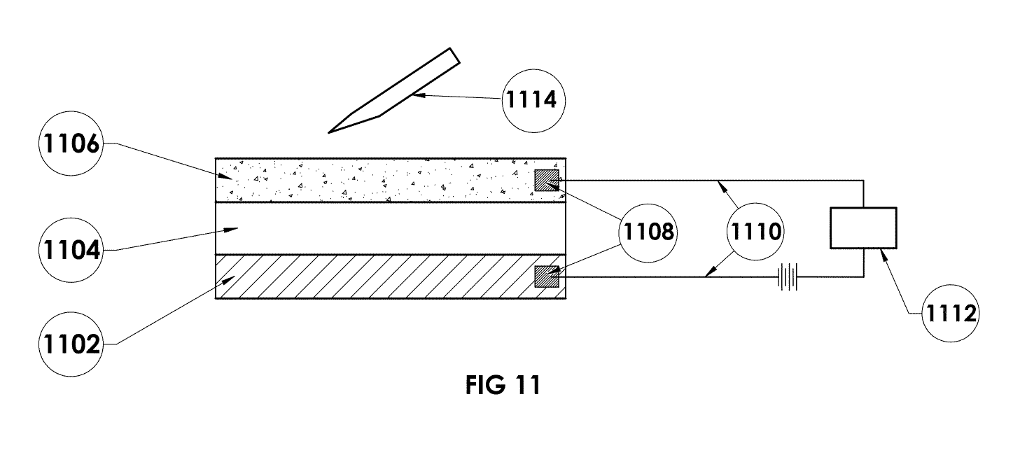

FIG. 11 is a depiction of a multilayer sensor 1100 that illustrates an embodiment of the invention whereby a metallic, or electrical conductive tool or probe, such as a scalpel or needle 1114, is inserted into a flexible substrate consisting of a highly electrical composite rubber 1106, or a dielectric material which the probe 1114 will penetrate, an electrically insulated layer 1104 which is between the material 1106 and the carbon nanotube sensor 1102 whereby the carbon nanotube sensor is comprised of, for example, conductive nantubes such as carbon nanotubes dispersed within a flexible or elastic electrically insulating material such as rubber. Electrodes 1108 are affixed to the electrically conductive layer 1106 and to the carbon nanotube polymer layer 1102. Upon penetration of the electrically conductive tool 1114 of both the electrically conductive layer 1106 and the sensor layer 1104 the circuit is complete and the electrical response is transmitted through connections 1110 to a control and data collection point 1112.

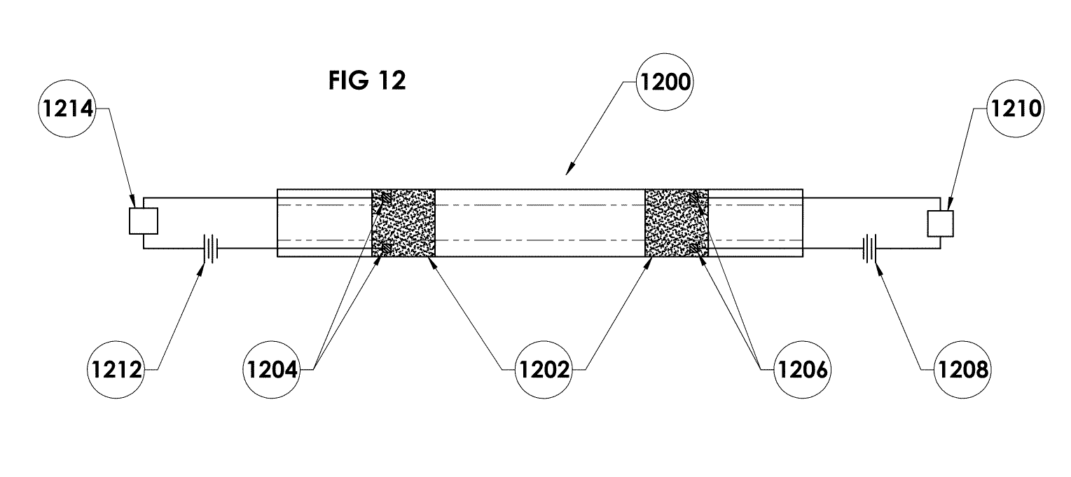

FIG. 12 is a representation of a medical training device 1200, such as synthetic vein or artery, wherein piezoresistive sensors 1202 are embedded within the body of the device. Attached to the sensors 1202 are electrodes 1204 and 1206 to which are connected to a voltage source 1208 and 1212. Response of the sensor is then displayed in a control unit 1210 and 1212. The medical training device will respond to pressure, elastic deformation and even surface penetration.

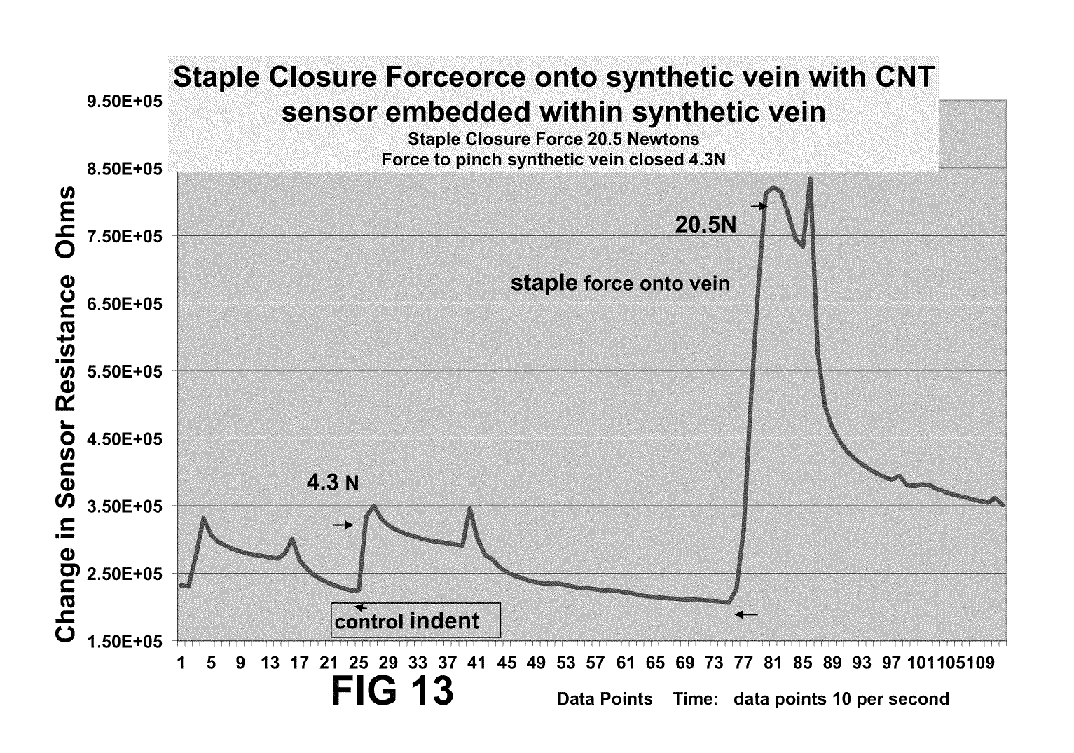

FIG. 13 is the responsive force plot of the force of a staple applied to a synthetic vein of FIG. 12. In this plot, a control force of 4.3 Newtons, identified as the control indent, is the force necessary to compress the synthetic vein to a closed position at the position of the piezoresistive sensor 1202 in FIG. 12. The force of applying a staple to the synthetic vein by a medical stapling device was measured by the sensor at 20.5 Newtons. Sensitivity of the system has been clearly evidenced in practice on various structures as having substantive threshold of 0.1 Newtons, and lower threshold levels of 0.05 have also been evidenced. An upper limit is not necessary for practice of this technology as there are natural levels of pressure that would never be exceeded.

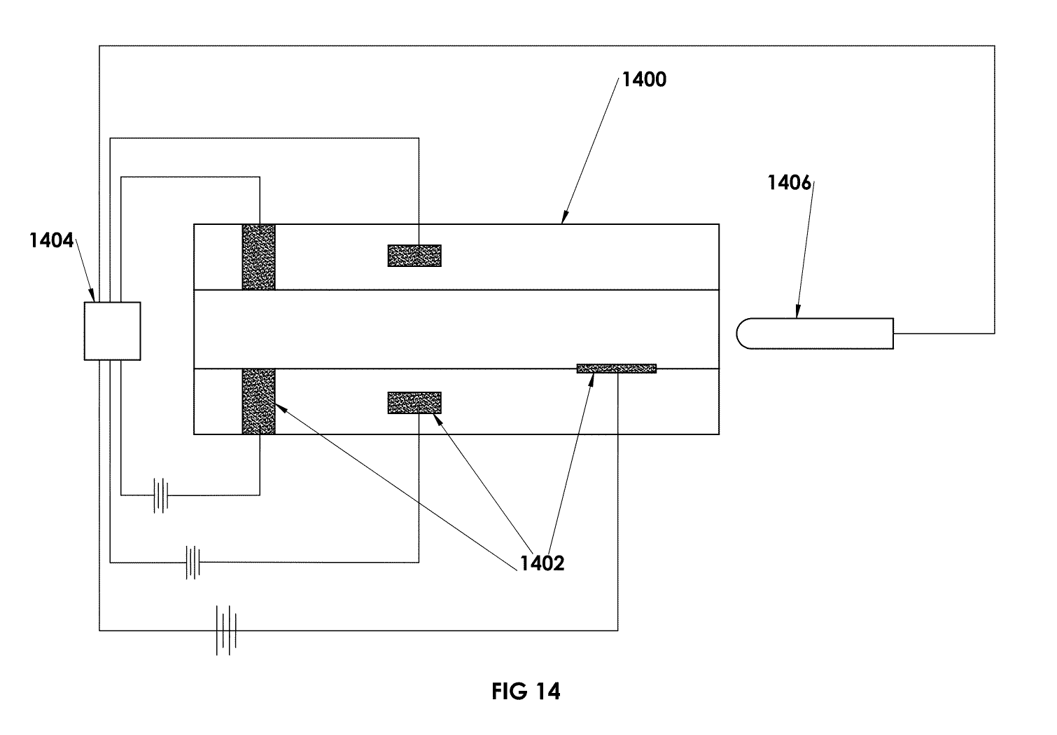

FIG. 14 exemplifies an application of the sensors applied in multiple regions of a model employing various aspects of the invention. This figure is a sectioned view of a body 1400 which may represent a model of a body part such as an esophagus, colon or other organ into which a diagnostic or therapeutic probe may be inserted. Within body 1400 are sensors 1402 which may be disposed onto the interior diameter surface, the outer diameter surface or in between. Probe 1406 may be an optical probe, a clasping device, a balloon device, a device with electrical conductivity, any medically functional device or combinations found within the medical field. In this depiction, as an example, probe 1406 has an electrical component which upon touching a sensor 1402 disposed on the interior diameter surface, will complete the sensor circuit giving information on initial physical contact with the carbon nanotube component of the sensor. This type of sensing is similar to the embodiment depicted in FIG. 11. As the probe 1406 continues through the model, forces it exerts on the model will be sensed by sensors 1402 and displayed on the control unit 1404.

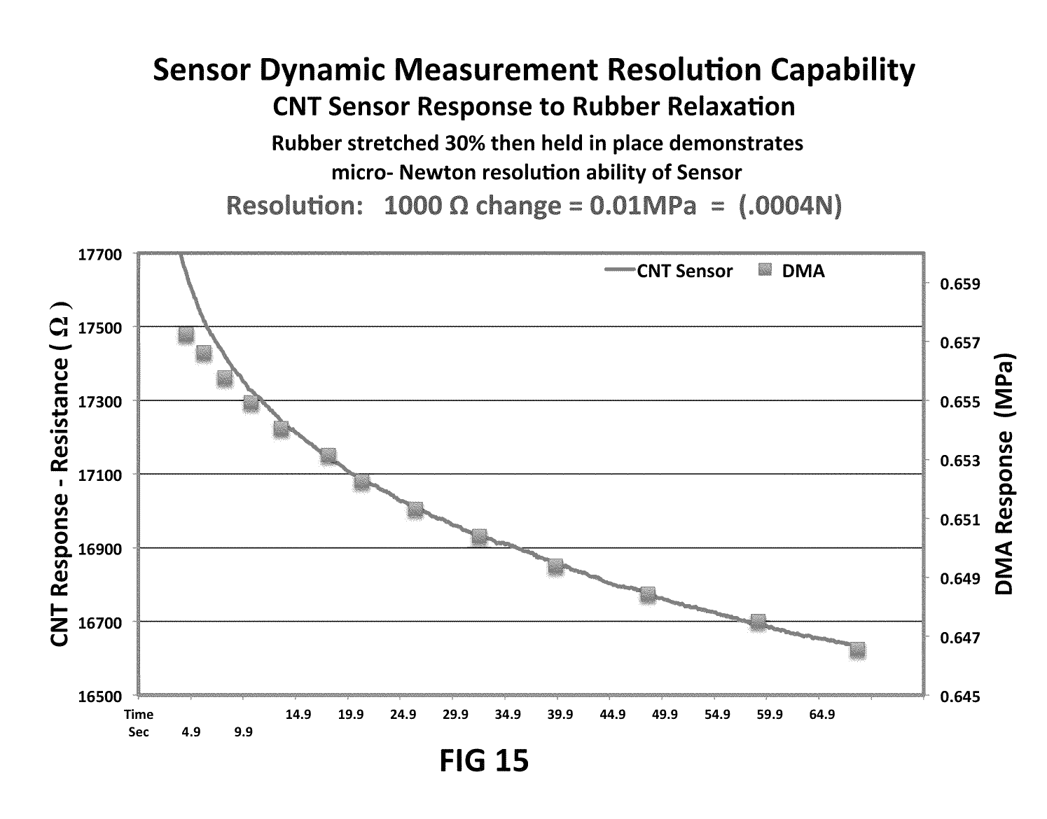

FIG. 15 is the dynamic response of a carbon nanotube sensor embedded within a rubber matrix as compared with the dynamic response of a dynamic mechanical analysis (DMA) measurement device. The plot show the CNT sensor response as a solid line and the DMA response as squares. In this test the rubber was stretched 30% elongation and held. The response seen is that of the stress-relaxation of the rubber with the sensor embedded within. The plot demonstrates the resolution of the CNT sensor to be 40 micro Newtons or less.

To achieve desired or designed electrical properties to a polymer or elastomer as described herein, such as an epoxy resin, elastomeric polymer or rubber, addition of moderate percentages, such as between 0.5% up to 4% by total weight of the polymer of conductive nanoparticles and especially carbon nanoparticles may be used. Loading with larger conductive particles such as carbon black at levels above 10% by total weight of the composition or total weight of the elastomer, often result in compromised physical properties such as hardness, tensile, thermal and compression. In addition, the electrical conductivity is negatively altered upon large deformations of the material to the point whereby electrical contact between the conducting particles is broken. The addition of very small amounts, even less than 2% by total weight of the composition (as described herein), of carbon nanotubes increases the electrical conductivity of the base material while preserving desired physical properties of the original polymer. The relatively lower loading of carbon nanotubes to a silicone rubber elastomer preserve desired original liquid silicone rubber physical properties such as hardness, tensile, elongation and compression. Low loading, by weight, of carbon nanotubes to a base polymer significantly changes the electrical properties. For example, a 0.5% or 1.0% loading of multi-wall carbon nanotubes dispersed into a liquid polymerizable to a silicone rubber, changes the resistivity of the original silicone rubber elastomer from 10¹³Ω cm to 10³Ω cm, with no significant change in the other important properties of the original properties. Additionally, large deformations of the nanotube composite do not negatively affect the electrical conductance of the material rather the electrical conductivity is maintained.

Also considered within the scope of this disclosure are: types of sensor devices and/or systems used to determine and/or measure strain or pressure. The sensors are used to determine and/or measure the amount of pressure or strain applied to an associated surface and used to determine and/or measure tissue thickness, and to determine or measure pressure and/or to provide pressure or strain data to a processor which correlates the pressure data with tissue thickness using a look-up table or other data structure. By knowing the strain or pressure data, a surgeon or technician can then determine the proper alignment of the device before completing the medical procedure.

The processor may be housed in a remotely programmable apparatus which also includes a memory for storing the script programs and the responses to voltage data flow. The remotely programmable apparatus may further include a microprocessor connected to the wires (effectively the communication device from the sensor, with or without a preamplifier), a user interface, and the memory. The microprocessor executes the script programs to identify the strain, communicate the results sets to the practitioner (e.g., through a monitor or printed output or audio signal), receive possible responses to the results of the data (e.g., a signal to readjust the device or reduce the exhibited strain), and transmit the responses to the server and/or monitor through communication networks.

The system may also include wireless communication between the voltage meter reading sensor output and the processor. For example, a microprocessor may be preferably connected to memory using a standard two-wire I²C interface or using a wireless connection. The microprocessor is also connected to user input buttons to initiate activity, alter read-outs requested, respond to signals from the sensor, start a print-out, and the like (as through an I/O port or dedicated printer port, LED, a clock and a display driver. The clock could indicate the current date and time to the microprocessor and measure duration of strain or pressure. The clock may be a separate component, but is preferably built into microprocessor. The display driver operates under the control of microprocessor to display information on a video display or monitor. The microprocessor may be any microprocessor in any format, including a laptop (PC or Mac) and operate on any operating system, including Linux. For example, a PIC 16C65 processor which includes a universal asynchronous receiver transmitter (UART) is an example of a useful processor for communicating with a modem and a device interface. A CMOS switch under the control of the microprocessor alternately connects modem and interface to the UART.

For the purposes of the implementation of the invention, a study was conducted using very low loadings of carbon nanotubes in an elastomeric liquid silicone rubber polymer. The resultant data concluded that desirable electrical properties were conferred to the liquid silicone rubber elastomeric polymer with relatively low, e.g., less than 4% or less than 3%, loadings of multi-walled carbon nanotubes. In addition, the study showed that the desired physical properties were maintained, and that no diluent behavior was observed. Further, the study showed that uniform resistivity was achieved throughout the liquid silicone carbon nanotube rubber composite. These conclusions support the inference that a liquid silicone carbon nanotube rubber composite can be effectively designed as an electrically conductive elastomeric material, while maintaining desirable physical properties such as tensile strength, elongation to break, compression and hardness.

Conventional and nano static and dynamic properties testing of materials, such as tensile, elongation, compression set, Dynamic Mechanical Analysis, surface and volume resistivity, etc., are often used to characterize material properties. Values from these tests are considered in the choice of materials suitable for application in the flexible sensor. Such test were conducted on carbon nanotube liquid silicone rubber composites to evaluate the effect of different loadings of carbon nanotube with different liquid rubbers.

In addition for the purpose of the invention, a study was conducted using very low loading of carbon nanotubes in an elastomeric silicone rubber polymer, measuring the changes in the electrical resistivity of the composite polymer during deformation. The changes in resistivity were measured as a function in the change of the output current of the material with a constant voltage applied to the material. The study compared loadings, by weight, of carbon nanotubes homogeneously mixed in the standard silicone polymers of between 0.5% and 2%. The resultant composites were deformed under various loading conditions and the change in resistivity of the composite monitored. For the purpose of the medical application, the study used voltages of between 0.01 and 1 volts. The study conducted measured large repeated deformations such as tensile strain in the order of 10 mm elongation as well as small deformation in the order of microns. The resultant change in resistivity correlated with the amount of deformation or force applied to the polymer composite. Although the term “constant voltage” is used, other electrical measurements are also used. For example, a constant current may be used (and voltage measured) It is also possible to use any other means such as resistive bridge circuit configurations or ballast circuits to determine resistance change.

Another aspect of the present technology includes accurate measurement of the amount of deformation of, strain exhibited on, or pressure exerted upon, an elastomeric medical device or component or sub-component inserted into a patient is determined by utilizing a sensor as described herein attached to or molded within the elastomeric medical device and exhibiting the above described piezoresitive properties that conductive nanotubes confer to an elastic medium. Such a sensor can be used to measure elongation or strain of a medical device during insertion, or immediately after insertion or even long after insertion into the patient. Such a sensor can also measure the deformation or load that is placed upon the medical device by the organ or with the body part with which the medical device is in contact. That measurement may be a direct pressure measurement, or by comparing strain with known degrees of pressure applied perpendicular to the sensor (and using a look-up table). Such a sensor may also be used to measure the amount of pressure that is being applied to a body part by the medical device. Such a sensor may also be used to monitor changes over time of the elongation, deformation, strain, load or pressure of an object or body part to which the sensor is affixed.

The present invention also relates to an electrically conductive rubber whereby the conductive agent applied to a flexible polymer base may be carbon nanotubes. The carbon nanotubes loadings are dispersed homogeneously into the polymer base such that the flexibility of the original base polymer is not dramatically compromised, and such that the electrical response of the composite is not significantly compromised (e.g., by more than 15%) over repeated deformations (e.g., over 20 deformations with greater than 100% elongation). A constant voltage is applied to the sensor and the electrical current is monitored at a point some distance from the voltage input through electrical connection with the electrodes or wires on the sensor. As the sensor is deformed, the current will change in response to the deformation due to the change in electrical resistivity of the composite material. For sensing deformation in devices in medical applications, the input voltage may be very low, in the order of less than 1 volt (e.g., 0.05 up to 1 volt), depending upon the electrical conductivity of the composite polymer. For medical applications the nanotube composite may be incased within a flexible polymer to insulate the electrically conductive composite and to comply with FDA regulations that may concern nano particle exposure.

The invention further relates to a sensor for which elongation and/or stress of the sensor is directly related to the distance that the sensor, or the medical device to which the sensor is affixed, is pulled, stressed, flexed, expanded or compressed. The distance may be a continuous pull, inflated expansion or compression or an incremental pull, stress, inflated expansion or compression of the sensor. The change in resistivity of the nanotube composite sensor directly correlates to the change in distance that the sensor is pulled, stressed, flexed or compressed. The change in resistivity may be measured directly as a change in resistance or as the change in current when a constant voltage is applied. Additionally, the load placed upon the sensor, or the medical device to which it is affixed or molded within, can be determined likewise by the change in resistivity of the nanotube composite sensor.

Various other aspects of the invention also relate to a flexible electrically conductive nanotube silicone rubber composite that is contained within a non electrically conductive medical grade silicone rubber, for the express purpose of distance, inflated expansion, compression or load measurement by observing the change in electrically resistivity of the nanotube composite. The attaching element can be used to attach the sensor directly to other sensors or devices attached to a medical patient for the purpose of measuring the stress or strain or other applied forces to the device. Additionally a sensor is described having at least an elastic body containing conductive nanotubes homogeneously distributed therein, the sensor contained or attached to or molded within an elastic body not containing conductive nanotubes and not electrically conductive, of which at least one surface of the sensor with physical attaching element thereon. Where embedded in another material, the attaching members assure elongation along with the embedding body.

The present technology using the novel sensors described and enabled herein may also be used within the field of real or training medical tools. The compositions of the sensors and layers in which they are carried may be the same as those described above. In the tool technology, more rigid substrates may also be used, as many tool are composed of more rigid components, compositions and layers, such as metal, ceramics, polymers (e.g., especially thermoset rigid polymers) composite materials and the like. Where the rigid substrate is conductive (e.g., metallic or conductive polymers or composites), there should be some electrically insulating material between the conductive nanotube/nanoparticle components of the sensor and the carrying layer (e.g., the stretchable, flexible, compressible layer) and the conductive substrate. In many cases the insulating capability of the elastomeric component may be sufficient to this end, but additional electrical insulation may also be desirable.

The general functions and structures of the responsive tool technology may be generally described as a responsive tool having a major surface and a sensor attached to and aligned with the major surface of the responsive tool, wherein:

the sensor comprises an elastic body containing conductive nanotubes homogeneously distributed therein to form a conductive path and at least two electrodes in electrical connection with the conductive path;

one of the electrodes has an external communication link for transmission of electrical transmission from the one of the electrodes; and

wherein at least compression on the elastic body alters electrical conductive properties of the elastic layer as a result of the compression. The at least two electrodes of the sensor should be in communication with both a power source and a processor, and the processor is configured to execute code to correlate variation in electrical signals through the elastic layer resulting from altered electrical conductive properties with forces applied to the elastic body of the responsive tool. The software in the processor used in executing the code may, as described elsewhere herein, contain a look-up table or may be initially calibrated for the test or practice procedures to which the tool would be used. The responsive tool may have the sensor adhered to the major surface or embedded in the major surface. The major surface my be a non-conductive composition having the conductive nanotubes therein. The major surface may be an interior or exterior surface on a responsive tool having a surface which is used to apply compressive forces to non-tool surfaces. If an interior surface, the applied pressure from the device is physically transmitted through components to the sensor. For example, where grips of forceps are used, the sensor may be at a lever fulcrum, leverage point or even grip of the device, while the forces applied to a distal object are at the opposed faces of the grip or forceps. The major surface may be a rigid surface and the elastic body of the sensor is a compressive surface composed of a rubber with hardness between 10 and 60 Asker C hardness. The responsive tool may have the major surface as part of an interior or exterior surface on a responsive tool having a surface which is used to penetrate or cut tissue. Again, the sensor need not be on the surface, but a sensor within the grip such that pressure on the blade or needle transmits forces to the sensor in the grip may be used. In designing the responsive tool, the tool may have opposed gripping surfaces and the sensor is located on at least one or both of the opposed gripping surfaces. The responsive tool may have the two electrodes of the sensor are in communication with both a power source and a processor, and wherein the processor is configured to execute code to correlate variation in electrical signals through the elastic layer resulting from altered electrical conductive properties with forces applied to the elastic body of the responsive tool. The tool with the sensor may have an elastic body of a silicone rubber containing a loading of between 0.5% and 3%, by total weight of conductive nanotubes. The responsive tool may have an electrically conductive silicone rubber composite comprised of a liquid silicone rubber with a multi-wall carbon nanotube loading of between 1%-3% by weight and a hardness between 10 and 60 Asker C hardness.

The responsive tool may be used in a method of detecting stress, pressure, contact, penetration or dimensional changes during use of a tool during a simulation of a procedure within an environment comprising positioning within the environment a responsive tool having a major surface and a sensor attached to and aligned with the major surface of the responsive model, the sensor comprises an elastic body containing conductive nanotubes homogeneously distributed therein to form a conductive path and at least two electrodes in electrical connection with the conductive path;
applying a current across the sensor through one of the at least two electrodes;
simulating activity within the environment imitating activity occurring during the medical procedure;
determining changes in the current or voltage; and
providing signals indicating changes in the current to a processor; and
the processor executing code to correlate determined changes in the current to stress, pressure, contact, penetration or dimensional changes in the responsive tool comprising the sensor.

The method may, for example, use a tool where the major surface comprises an interior or exterior surface on a responsive tool having opposed surfaces used to apply pressure to objects or the major surface is on at least one of two opposed surfaces on the tool and the procedure is a medical procedure. Again in the method, signals of determined changes correlated by the processor are provided by the processor in the form of image signals and the image signals are display in real-time on a visual display screen.

Another aspect of the technology includes a sensor comprising of an elastic body comprised of a silicone rubber containing a loading of between 0.25% and 5% (even up to 7% by weight) by weight or between 0.5% and 4%, by wt. of conductive nanotubes such as carbon nanotubes, homogeneously distributed therein, with electrodes adhered to or molded within the nanotube composite for the purpose of applying an electrical current through the composite and a detection system that detects absolute amounts of voltage and/or changes in voltage across the electrodes.

A further aspect of the present technology may include a sensor having an elastic body comprised of a liquid silicone rubber containing a loading of between 0.5% and 3%, by wt. of carbon nanotubes, homogeneously distributed therein, with electrodes adhered to or molded within the nanotube composite and contained entirely within a medical grade non conductive flexible silicone rubber.

Another aspect of the present invention may include an electrically conductive silicone rubber composite comprised of a liquid silicone rubber with a multi-wall carbon nanotube loading of between 1%-3% by weight and a hardness between 10 and 60 Asker C hardness.

An electrically conductive silicone rubber composite may have a liquid silicone rubber with a multi-wall carbon nanotube loading of between 0.5%-3% by weight, a hardness of between 10 and 60 Asker C and elongation property greater than 200%.

An electrically conductive silicone rubber composite may have a liquid silicone rubber with a multi-wall carbon nanotube loading of between 1%-3% by weight, a hardness of between 10 and 60 Asker C, an elongation property greater than 200% and electrical resistivity of 10³ Ohm/sq or less.

Although specific dimensions, compositions, voltages, materials and fields of use are described herein, it must be understood that these are examples enabling the generic scope of the invention and should not limit the scope of enforcement of claims herein.

What is claimed:

1. A responsive tool having a major surface and a sensor attached to and aligned with the major surface of the responsive tool, wherein:
the sensor comprises an elastic body containing conductive nanotubes homogeneously distributed therein to form a conductive path and at least two electrodes in electrical connection with the conductive path;
one of the electrodes has an external communication link for transmission of electrical transmission from the one of the electrodes; and
wherein at least compression on the elastic body alters electrical conductive properties of the elastic layer as a result of the compression.

2. The responsive tool of claim 1 wherein the at least two electrodes of the sensor are in communication with both a power source and a processor, and wherein the processor is configured to execute code to correlate variation in electrical signals through the elastic layer resulting from altered electrical conductive properties with forces applied to the elastic body of the responsive tool.

3. The responsive tool of claim 2 wherein the sensor adhered to the major surface or embedded in the major surface.

4. The responsive tool of claim 2 wherein the major surface comprises a non-conductive composition having the conductive nanotubes therein.

5. The responsive tool of claim 1 wherein the major surface comprises an interior or exterior surface on a responsive tool having a surface which is used to apply compressive forces to non-tool surfaces.

6. The responsive tool of claim 2 wherein the sensor has a capability of sensing pressure against the sensor at least at forces of 0.5 Newtons against a surface of the sensor.

7. The responsive tool of claim 1 wherein the major surface comprises an interior or exterior surface on a responsive tool having a surface which is used to penetrate or cut tissue.

8. The responsive tool of claim 7 wherein the responsive tool has opposed gripping surfaces and the sensor is located on at least one of the opposed gripping surfaces.

9. The responsive tool of claim 7 wherein the responsive tool has opposed gripping surfaces and the sensor is located on each of the opposed gripping surfaces.

10. The responsive tool of claim 5 wherein the two electrodes of the sensor are in communication with both a power source and a processor, and wherein the processor is configured to execute code to correlate variation in electrical signals through the elastic layer resulting from altered electrical conductive properties with forces applied to the elastic body of the responsive tool.

11. The responsive model of claim 6 wherein the two electrodes of the sensor are in communication with both a power source and a processor, and wherein the processor is configured to execute code to correlate variation in electrical signals through the elastic layer resulting from altered electrical conductive properties with forces applied to the elastic body of the responsive tool.

12. The responsive model of claim 9 wherein the two electrodes of the sensor are in communication with both a power source and a processor, and wherein the processor is configured to execute code to correlate variation in electrical signals through the elastic layer resulting from altered electrical conductive properties with forces applied to the elastic body of the responsive tool.

13. The sensor of claim 1 wherein the sensor comprises an elastic body of a silicone rubber containing a loading of between 0.5% and 3%, by total weight of conductive nanotubes.

14. The responsive model of claim 13 wherein at least two electrodes of the sensor are in communication with both a power source and a processor, and wherein the processor is configured to execute code to correlate variation in electrical signals through the elastic layer resulting from altered electrical conductive properties with forces applied to the elastic body of the responsive tool.

15. The responsive model of claim 12 wherein the sensor comprises an electrically conductive silicone rubber composite comprised of a liquid silicone rubber with a multi-wall carbon nanotube loading of between 1%-3% by weight and a hardness between 10 and 60 Asker C hardness.

16. A method of detecting stress, pressure, contact, penetration or dimensional changes during use of a tool during a simulation of a procedure within an environment comprising positioning within the environment a responsive tool having a major surface and a sensor attached to and aligned with the major surface of the responsive model, the sensor comprises an elastic body containing conductive nanotubes homogeneously distributed therein to form a conductive path and at least two electrodes in electrical connection with the conductive path;
applying a current across the sensor through one of the at least two electrodes;
simulating activity within the environment imitating activity occurring during the medical procedure;
determining changes in the current or voltage; and
providing signals indicating changes in the current to a processor; and
the processor executing code to correlate determined changes in the current to stress, pressure, contact, penetration or dimensional changes in the responsive tool comprising the sensor.

17. The method of claim 16 wherein the major surface comprises an interior or exterior surface on a responsive tool having opposed surfaces used to apply pressure to objects.

18. The method of claim 17 wherein the major surface is on at least one of two opposed surfaces on the tool and the procedure is a medical procedure.

19. The method of claim 18 wherein signals of determined changes correlated by the processor are provided by the processor in the form of image signals and the image signals are display in real-time on a visual display screen.
